(19) United States
(12) Patent Application Publication
(10) Pub. No.: US 2021/0393216 A1
(43) Pub. Date: Dec. 23, 2021

(54) MAGNETIC RESONANCE SYSTEM, IMAGE DISPLAY METHOD THEREFOR, AND COMPUTER-READABLE STORAGE MEDIUM

(71) Applicant: GE Precision Healthcare LLC, Wauwatosa, WI (US)

(72) Inventors: Nan CAO, Beijing (CN); Yongchuan LAI, Beijing (CN); Jianyong YIN, Beijing (CN)

(21) Appl. No.: 17/304,359

(22) Filed: Jun. 18, 2021

(30) Foreign Application Priority Data
Jun. 23, 2020 (CN) 202010582255.9

(51) Int. Cl. A61B 5/00; A61B 5/055; G01R 33/56

(52) U.S. Cl. CPC A61B 5/742; G01R 33/5602; A61B 5/7264; A61B 5/055

(57) ABSTRACT

Embodiments of the present invention provide a magnetic resonance system, an image display method thereof, and a computer-readable storage medium. The method includes: acquiring sequential images to be displayed, the sequential images comprising a plurality of images; determining an identical window width for the plurality of images; and displaying the plurality of images of the sequential images based on the window width.

### CROSS-REFERENCE TO RELATED APPLICATIONS

This application claims priority pursuant to 35 U.S.C. 119(a) to China Patent Application No. 202010582255.9 filed on Jun. 23, 2020, the disclosure of which is herein incorporated by reference in its entirety.

### TECHNICAL FIELD

Embodiments disclosed in the present invention relate to medical imaging technologies, and more particularly relate to a magnetic resonance system, an image display method therefor, and a computer-readable storage medium.

### BACKGROUND

In the prior art, the magnetic resonance imaging (MRI) technology can be used to perform imaging on human tissues to obtain a plurality of cross-sectional images, i.e., an image sequence, of a region of interest. At a certain stage of magnetic resonance examination, the image sequence needs to be displayed on the human-computer interaction interface of a magnetic resonance system in a certain arrangement manner, so as to facilitate reading and viewing operations by physicians. In practical applications, when reading sequential images, physicians often need to draw on experience to manually adjust display parameters, for example, a window width, of one or more images in the image sequence because of great brightness differences between various images in the image sequence or of an unsatisfactory display effect for clinical diagnosis. This manner affects the efficiency of magnetic resonance imaging-based diagnosis.

### SUMMARY

An embodiment of the present invention provides an image display method for a magnetic resonance system, the method comprising:

acquiring sequential images to be displayed, the sequential images comprising a plurality of images;

determining an identical window width for the plurality of images; and

displaying the plurality of images of the sequential images based on the window width.

In one embodiment, the determining an identical window width for the plurality of images further comprises:

acquiring a sorting result of pixel values of the plurality of images; and

determining the window width based on a pixel value at a preset ordinal position in the sorting result or a plurality of pixel values within a preset ordinal position range.

In one embodiment, the window width is determined based on a pixel value at an intermediate ordinal position in the sorting result or a plurality of pixel values within an intermediate ordinal position range containing the intermediate ordinal position.

In one embodiment, the determining the window width based on the plurality of pixel values within the intermediate ordinal position range comprises: determining an average of the plurality of pixel values as the window width.

The method may further comprise: determining an adjustment factor, and adjusting the window width based on the adjustment factor. In one embodiment, the plurality of images are displayed based on the adjusted window width.

In one embodiment, the adjustment factor is determined based on one or more pieces of imaging information corresponding to the sequential images.

The one or more pieces of imaging information comprise one or more of an imaging site, a scan sequence, a scan plane, an echo time, an inversion time, and a repetition time configured by the magnetic resonance system when generating the sequential images.

In one embodiment, the one or more pieces of imaging information are input into a predetermined deep learning network, and the adjustment factor is output through the deep learning network.

In one embodiment, the step of determining the adjustment factor comprises:

determining user information for displaying the sequential images; and determining a corresponding adjustment factor based on the determined user information.

In one embodiment, the determined user information is input into a predetermined second deep learning network, and the adjustment factor is output through the second deep learning network.

Another embodiment of the present invention further provides a magnetic resonance system, comprising:

a scanner configured to generate sequential images by performing magnetic resonance scanning on an imaging site, the sequential images comprising a plurality of images;

a processor configured to acquire the sequential images and determine an identical window width for the plurality of images; and a display unit displaying the plurality of images of the sequential images based on the identical window width.

Another embodiment of the present invention further provides a computer-readable storage medium for storing computer-readable instructions, wherein the computer-readable instructions are configured to perform the image display method according to any one of the embodiments described above.

It should be understood that the brief description above is provided to introduce, in a simplified form, some concepts that will be further described in the Detailed Description. The brief description above is not meant to identify key or essential features of the claimed subject matter. The protection scope is defined uniquely by the claims that follow the detailed description. Furthermore, the claimed subject matter is not limited to implementations that solve any disadvantages noted above or in any section of the present disclosure.

### BRIEF DESCRIPTION OF THE DRAWINGS

The present invention will be better understood by reading the following description of non-limiting embodiments with reference to the accompanying drawings, where

FIG. 1 shows a schematic structural diagram of a magnetic resonance system;

FIG. 2 shows a flowchart of an image display method according to an embodiment of the present invention;

FIG. 3 shows a flowchart of an image display method according to another embodiment of the present invention;

FIG. 4 shows a flowchart of an image display method according to another embodiment of the present invention;

FIG. 5 shows a flowchart of an image display method according to another embodiment of the present invention;

FIG. 6 shows a flowchart of an image display method according to another embodiment of the present invention;

FIG. 7 shows a flowchart of an image display method according to another embodiment of the present invention; and

FIG. 8 shows a flowchart of an image display method according to another embodiment of the present invention.

### DETAILED DESCRIPTION

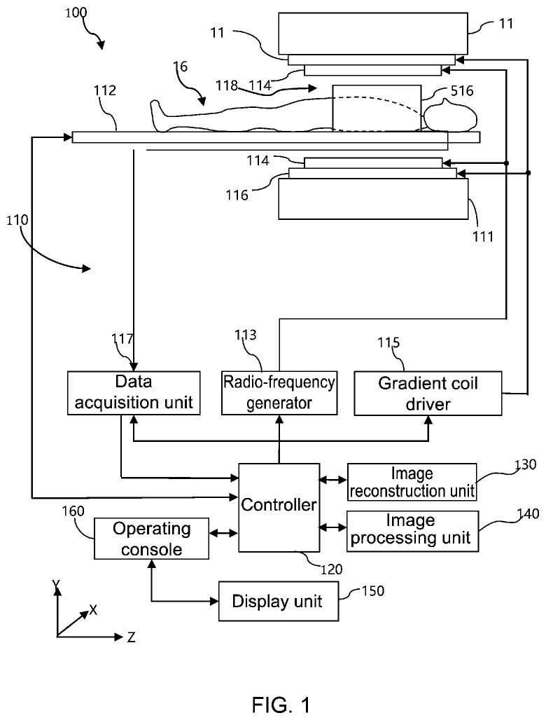

FIG. 1 shows a schematic structural diagram of a magnetic resonance system, and the magnetic resonance system 100 comprises a scanner 110. The scanner 110 is configured to perform magnetic resonance scanning on an object (for example, a human body) 16 to generate image data of a region of interest of the object 16, and the region of interest may be a predetermined imaging site or tissue to be imaged. The image data may be sequential images having a plurality of images. In one embodiment, the plurality of images may be two-dimensional images corresponding to a plurality of cross-section (or tomographic) positions of the region of interest.

The magnetic resonance system may include a controller 120, which is coupled to the scanner 110 to control the scanner 110 to perform the aforementioned magnetic resonance scanning process. Specifically, the controller 120 may send a sequence control signal to relevant components (such as a radio-frequency generator and a gradient coil driver that will be described below) of the scanner 110 through a sequence generator (not shown), so that the scanner 110 performs the preset scan sequence.

Those skilled in the art could understand that the “scan sequence” refers to a combination of pulses having specific amplitudes, widths, directions, and timings that are applied while performing a magnetic resonance imaging scan. The pulses may typically include, for example, a radio-frequency pulse and a gradient pulse. The radio-frequency pulse may include, for example, a radio-frequency transmit pulse for exciting protons in the human body to resonate, and the gradient pulse may include, for example, a slice selection gradient pulse, a phase encoding gradient pulse, and a frequency encoding gradient pulse. Typically, a plurality of scanning sequences may be pre-configured in the magnetic resonance system, so that a sequence adapted for clinical testing requirements is selectable. The clinical testing requirements may include, for example, an imaging site, an imaging function, etc.

In practice, it may be required to select different scan sequence types depending on different clinical applications, for example, an echo planar imaging (EPI) sequence, a gradient echo (GRE) sequence, a spin echo (SE) sequence, a fast spin echo (FSE) sequence, a diffusion-weighted imaging (DWI) sequence, an inversion recovery (IR) sequence, and the like; and in different clinical applications, each scan sequence may have different scan sequence parameters, for example, a T1-weighted value, a T2-weighted value, an echo time, a repetition time, an inversion recovery time, etc.

In an example, the scanner 110 may include a main magnet assembly 111, a table 112, a radio-frequency generator 113, a radio-frequency transmitting coil 114, a gradient coil driver 115, a gradient coil assembly 116, and a data acquisition unit 117.

The main magnet assembly 111 usually includes an annular superconducting magnet defined in a housing. The annular superconducting magnet is mounted in an annular vacuum container. The annular superconducting magnet and the housing thereof define a cylindrical space, for example, a scanning chamber 118 shown in FIG. 1, surrounding the object 16. The main magnet assembly 111 generates a constant magnetic field, i.e., a B0 field, in a Z direction of the scanning chamber 118. Typically, a uniform portion of the B0 field is formed in a central region of the main magnet.

The table 112 is configured to carry the object 16, and travel in the Z direction to enter or exit the scanning chamber 118 in response to the control of the controller 120. For example, in an embodiment, an imaging volume of the object 16 may be positioned at a central region of the scanning chamber with uniform magnetic field strength so as to facilitate scanning imaging of the imaging volume of the object 16.

The magnetic resonance system transmits a static magnetic pulse signal to the object 16 located in the scanning chamber by using the formed B0 field, so that protons in a resonance volume within the body of the object 16 precess in an ordered manner to generate a longitudinal magnetization vector.

The radio-frequency generator 113 is configured to generate a radio-frequency pulse, for example, a radio-frequency excitation pulse, in response to a control signal of the controller 120. The radio-frequency excitation pulse is amplified (for example, by a radio-frequency power amplifier (not shown)) and then applied to the radio-frequency transmitting coil 114, so that the radio-frequency transmitting coil 114 emits to the object 16 a radio-frequency field B1 orthogonal to the B0 field to excite nuclei in the aforementioned resonant volumes, and generate a transverse magnetization vector.

The radio-frequency transmitting coil 114 may include, for example, a body coil disposed along an inner circumference of the main magnet, or a head coil dedicated to head imaging. The body coil may be connected to a transmitting/receiving (T/R) switch (not shown). The transmitting/receiving switch is controlled so that the body coil can be switched between a transmitting mode and a receiving mode. In the receiving mode, the body coil may be configured to receive a magnetic resonance signal from the object 16.

After the end of the radio-frequency excitation pulse, a free induction decay signal, namely, a magnetic resonance signal that can be acquired, is generated in the process that the transverse magnetization vector of the object 16 is gradually restored to zero.

The gradient coil driver 115 is configured to provide a suitable current/power to the gradient coil assembly 116 in response to a gradient pulse control signal or a shimming control signal sent by the controller 120.

The gradient coil assembly 116, on one hand, forms a varying magnetic field in an imaging space so as to provide three-dimensional position information to the magnetic resonance signal, and on the other hand, generates a compensating magnetic field of the B0 field to shim the B0 field.

The gradient coil assembly 116 may include three gradient coils. The three gradient coils are respectively configured to generate magnetic field gradients inclined to three spatial axes (for example, X-axis, Y-axis, and Z-axis) perpendicular to each other. More specifically, the gradient coil assembly 116 applies a magnetic field gradient in a slice selection direction (Z direction) so as to select a layer in the imaging volume. Those skilled in the art understand that the layer is any one of a plurality of two-dimensional slices distributed in the Z direction in the three-dimensional imaging volume. When the imaging is scanned, the radio-frequency transmitting coil 114 transmits a radio-frequency excitation pulse to the layer of the imaging volume and excites the layer. The gradient coil assembly 116 applies a magnetic field gradient in a phase encoding direction (Y direction) so as to perform phase encoding on a magnetic resonance signal of the excited layer. The gradient coil assembly 116 applies a gradient field in a frequency encoding direction of the object 16 so as to perform frequency encoding on the magnetic resonance signal of the excited layer.

The data acquisition unit 117 is configured to acquire the magnetic resonance signal (for example, received by the body coil or a surface coil) in response to a data acquisition control signal of the controller 120. In an embodiment, the data acquisition unit 117 may include, for example, a radio-frequency preamplifier, a phase detector, and an analog/digital converter, where the radio-frequency preamplifier is configured to amplify the magnetic resonance signal, the phase detector is configured to perform phase detection on the amplified magnetic resonance signal, and the analog/digital converter is configured to convert the phase-detected magnetic resonance signal from an analog signal to a digital signal.

The magnetic resonance system 100 includes an image reconstruction unit 130, which may reconstruct, based on the aforementioned digitized magnetic resonance signal, a series of two-dimensional cross-sectional images of an imaging volume of the object 16, i.e., the image sequence. Specifically, the reconstruction unit may perform the image reconstruction described above based on communication with the controller 120.

The magnetic resonance system 100 includes a processing unit 140, which may perform any required image processing on any image in the image sequence, for example, image correction, image display parameter determination, etc. The image processing described above may be an improvement or adaptive adjustment made to an image in terms of any one of contrast, uniformity, sharpness, brightness, etc. Specifically, the processing unit 140 may perform the image processing described above based on communication with the controller 120.

In one embodiment, the controller 120, the image reconstruction unit 130, and the processing unit 140 may separately or collectively include a computer and a storage medium, and the storage medium records a predetermined control program or data processing program to be executed by the computer. For example, the storage medium may store a program configured to implement imaging scanning, image reconstruction, image processing, etc. For example, the storage medium may store a program configured to implement the image display method of the embodiments of the present invention. The storage medium may include, for example, a ROM, a floppy disk, a hard disk, an optical disk, a magneto-optical disk, a CD-ROM, or a non-volatile memory card.

The magnetic resonance system 100 may include a display unit 150, which may be configured to display an operation interface and various data or images generated during a data processing process. The display unit 150 may display, in response to a display control signal of the controller 120 (the display control signal may be generated in response to a request operation of a physician for reading images), the sequential images through the display unit 150 in a certain arrangement manner. For example, the images may be arranged according to the order of cross-section positions. In addition, the display unit 150 may also communicate with the processing unit 140 (for example, via the controller 120) to display the sequential images in accordance with display parameters determined by the processing unit 140.

When determining the display parameters of the sequential images, the processing unit 140 may specifically consider parameters related to contrast, sharpness, brightness, uniformity, etc., for example, a window width and a window level. Those skilled in the art would understand that the window width corresponds to an image pixel range, and the window level corresponds to the middle value in the pixel range. It is typically desirable to set the window level at a tissue most capable of reflecting a lesion (for example, a soft tissue, a bone, blood, etc.), and a window level closer to pixel values of the tissue yields better uniformity of the image. With that window level being set as the center, a smaller window width yields a higher contrast ratio.

The magnetic resonance system 100 includes a console 160, which may include a user input device, such as a keyboard, a mouse, etc. The controller 120 may communicate with the scanner 110, the image reconstruction unit, the processing unit 140, the display unit 150, etc, in response to a control command generated by a user based on operating the console 160 or an operation dashboard/button and the like disposed on a housing of a main magnet.

Typically, respective window widths and window levels are configured for all of the images, so that each image in the sequential images presented to the user is expected to have ideal image quality. For example, a corresponding window width is configured based on a maximum pixel value and a minimum pixel value of each image, and a window level is configured based on the window width. For example, the window level is a median value of the window width. However, in practice, a small amount of metal, vascular tissues, etc., in the human body might cause a high-brightness signal in a portion of an image, causing the maximum pixel value in the image to be excessively high, and leading to an excessively large window width. Accordingly, the obtained window level deviates significantly from pixel values of a tissue to be viewed, so that a portion of the images in the image sequence are too dark, and overall, large brightness differences within the image sequence are yielded, resulting in reading difficulties for physicians.

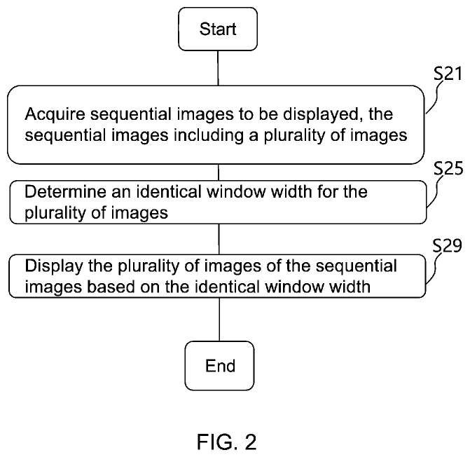

FIG. 2 shows a flowchart of an image display method according to an embodiment of the present invention. As shown in FIG. 2, the method includes steps S21, S23, and S25.

In step S21, sequential images to be displayed are acquired, where the sequential images include a plurality of images. For example, upon receiving a reading request of a user, the controller 120 may notify the processing unit 140 to retrieve a plurality of cross-sectional images generated from a magnetic resonance scan performed by the scanner 110 on a region of interest of the object 16, or to retrieve the plurality of cross-sectional images that have undergone image processing.

In step S25, an identical window width is determined for the plurality of images, and the determined window width is sent to the display unit 160 directly or through the controller 120.

In step S29, the plurality of images of the sequential images are displayed based on the identical window width. Specifically, when displaying the plurality of images, the display unit 160 may display pixel values outside of the pixel range defined by the window width as a background color, for example, the pixel values being “0”.

Setting an identical window width for a plurality of images in an image sequence prevents problems such as reading difficulties caused by excessive brightness differences between images under display, the need to perform corresponding manual adjustment on different images, etc. Setting a window level based on the identical window width also prevents the problem of poor uniformity between images.

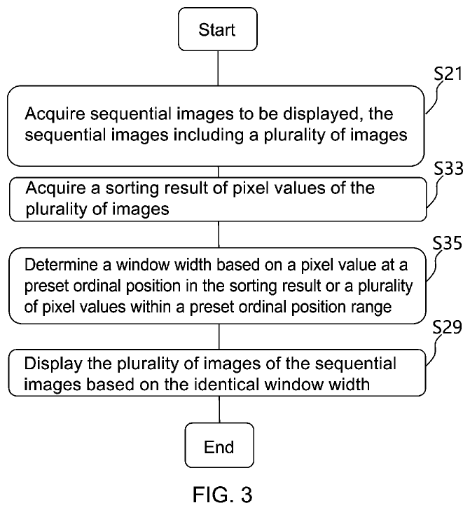

FIG. 3 shows a flowchart of an image display method according to another embodiment of the present invention. As shown in FIG. 3, the method includes steps S21, S33, S35, and S29.

In step S33, a sorting result of the pixel values of the plurality of images is acquired. For example, in the case that all of the pixel values of the plurality of images are counted to indicate that a total of N pixel values are included, then the N pixel values are sorted in a descending order or an ascending order (1, 2, 3 . . . m−2, m−1, m, m+1, m+2 . . . N), where m is a natural number between 1 and N.

In step S35, the window width is determined based on a pixel value at a preset ordinal position in the sorting result (for example, the m-th pixel value) or a plurality of pixel values within a preset ordinal position range (for example, from the (m−2)-th pixel value to the (m+2)-th pixel value, or the (q+)-th pixel value). Preferably, the preset ordinal position may be an intermediate ordinal position. For example, a window width may be determined based on a pixel value arranged at the intermediate ordinal position, or the window width may be determined based on a plurality of pixel values arranged within an intermediate range (i.e., an intermediate ordinal position range containing the intermediate ordinal position).

In a specific embodiment, the preset signal or the pixel value at the intermediate ordinal position may be directly set as the window width, or an average of the plurality of pixel values in a preset range or the intermediate ordinal position range may be set as the window width.

In this embodiment, a uniform window width may be configured for the plurality of images of the image sequence without complex computation. Moreover, since the selection is performed in accordance with the ordinal position of the pixel value(s) instead of merely relying on the maximum pixel value and the minimum pixel value, the problem of excessive image brightness or darkness caused by an excessively large or small maximum pixel value is prevented.

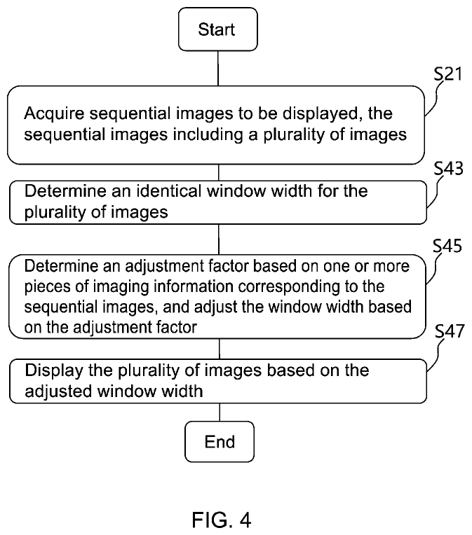

FIG. 4 shows a flowchart of an image display method according to another embodiment of the present invention. The image display method of this embodiment includes step S21, step S43, step S45, and step S47. Step S43 may be similar to step S25, for example, determining an identical window width for the plurality of images. More specifically, step S43 may also include steps S33 and S35 so as to determine the identical window width for the plurality of images.

In step S45, an adjustment factor is determined based on one or more pieces of imaging information corresponding to the sequential images, and the window width is adjusted based on the adjustment factor. The imaging information may include, for example, one or more of an imaging site, a scan sequence, a scan plane, an echo time, an inversion recovery time, and a repetition time configured by the magnetic resonance system when generating the sequential images.

Those skilled in the art would understand that: the imaging site may include a body part of a human body, for example, a head, an abdomen, a chest, a heart, and the like; the scanning plane is a scanning layer and corresponds to a two-dimensional cross-sectional image in an imaging sequence, and each scanning layer/plane has a specific cross-section position; the echo time refers to a time range from a radio-frequency excitation pulse to the center of an echo signal in the scanning sequence; the inversion time is a time range between the center of a 180° inversion pulse and the center of a 90° excitation pulse; and the repetition time is a time range between the centers of two adjacent excitation pulses.

The adjustment factor may be determined based on weights of one or more pieces of the imaging information described above. The adjustment described above may specifically include multiplying the adjustment factor with the window width. In a specific example, the adjustment factor may be less than or greater than 1.

In step S47, the plurality of images of the sequential images are displayed based on the determined window width, and specifically, the plurality of images are displayed based on the adjusted window width.

In this way, a suitable display manner may be selected for different imaging information to better meet clinical diagnostic requirements. For example, the adjustment factor may be different due to different imaging sites, since a higher image brightness may be required for reading images of one imaging site to make a lesion more readily observable, while a lower image brightness is required for reading images of another imaging site to make a lesion more readily observable. As another example, for the same imaging site, the brightness requirement for reading may also vary if a different scanning sequence or different scanning parameter is adopted, and a corresponding weight is used to determine a suitable window width adjustment factor to meet a corresponding requirement.

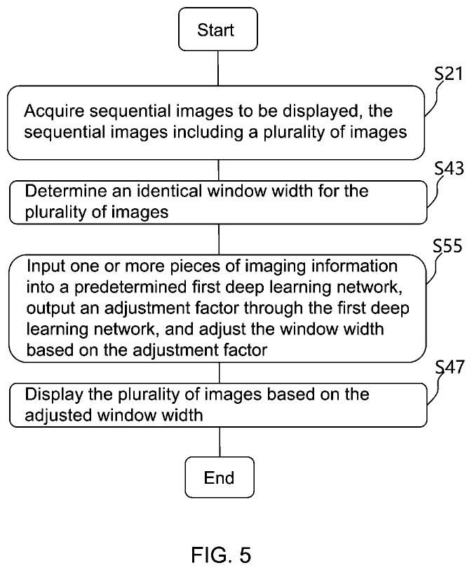

FIG. 5 shows a flowchart of an image display method according to another embodiment of the present invention.As shown in FIG. 5, the image display method of this embodiment includes step S21, step S43, step S55, and step S47. In step S55, one or more pieces of imaging information are input into a predetermined first deep learning network, an adjustment factor is output through the first deep learning network, and the window width determined in step S43 is adjusted based on the adjustment factor.

Data training may be performed by using the following exemplary method to obtain the first deep learning network described above.

In one step, ideal window width adjustment factors for a plurality of image sequences are acquired. For example, based on any of the above embodiments, initial window widths may be first determined for the respective image sequences, then the initial window widths may be adjusted manually, so that corresponding image sequences have ideal display brightness, then a plurality of window width adjustment factors corresponding to the plurality of image sequences are calculated based on the adjusted window widths and the initial window widths.

In another step, imaging information corresponding to the plurality of image sequences is determined, where the imaging information respectively corresponding to the respective image sequences may be one single type of information or different combinations of multiple types of information.

In another step, the plurality of window width adjustment factors are used as an output dataset, the imaging information corresponding to the plurality of image sequences is used as an input dataset, and a suitable machine learning network is selected to perform machine learning, so as to assign a network parameter associated with the input dataset and the output dataset to the machine learning network.

The trained first learning network enables, when displaying an image sequence, automatic acquisition of a window width adapted to imaging information thereof, so as to better meet the display requirement for clinical diagnosis.

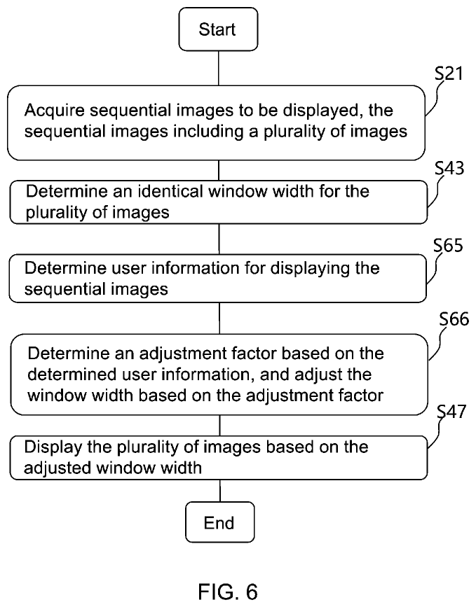

FIG. 6 shows a flowchart of an image display method of another embodiment of the present invention. As shown in FIG. 6, the image display method of this embodiment includes step S21, step S43, step S65, step S66, and step S47. In step S65, user information for displaying the sequential images is determined. In step S66, an adjustment factor is determined based on the determined user information, and the window width determined in step S43 is adjusted based on the adjustment factor. For example, a plurality of corresponding adjustment factors may be determined respectively based on multiple pieces of user information. In this way, when an image sequence is displayed, a window width adjustment factor may be automatically configured based on a personal habit or preference of a user to better meet a personalized display requirement of the user.

In this embodiment, the adjustment factor corresponding to user information may be stored in advance, and when a user performs a reading operation, information of the user is identified, so that the corresponding adjustment factor may be retrieved to adjust an initial window width.

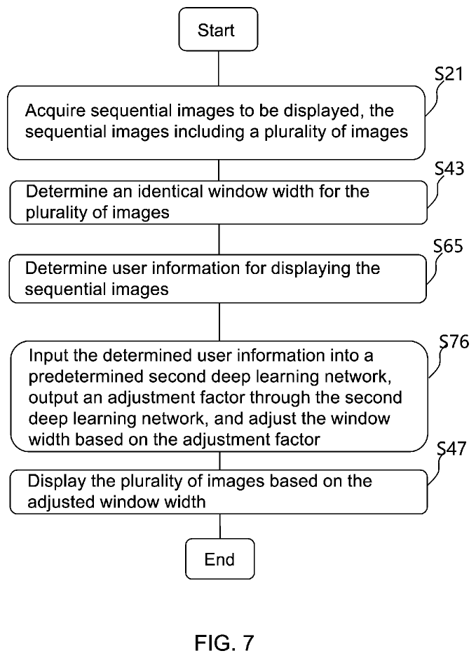

FIG. 7 shows a flowchart of an image display method of another embodiment of the present invention. As shown in FIG. 7, the image display method of this embodiment includes step S21, step S43, step S65, step S76, and step S47. In step S76, the determined user information is input into a predetermined second deep learning network, an adjustment factor is output through the second deep learning network, and the window width determined in step S43 is adjusted based on the adjustment factor.

Data training may be performed by using the following exemplary method to obtain the second deep learning network described above.

In one step, a plurality of window width adjustment factors for reading operations of a plurality of users may be acquired for one or a plurality of image sequences. For example, respective adjusted window widths during reading operations of the users and initial window widths prior to the reading operations may be acquired, and the window width adjustment factors corresponding to the users are calculated based on the adjusted window widths and the initial window widths.

In another step, the plurality of window width adjustment factors are used as an output dataset, the user information of the plurality of users is used as an input dataset, and a suitable machine learning network is selected to perform machine learning, so as to assign a network parameter associated with the input dataset and the output dataset to the machine learning network.

The trained second learning network enables, when displaying an image sequence, quick and accurate configuration of a window width adjustment factor based on a personal habit or preference of a user.

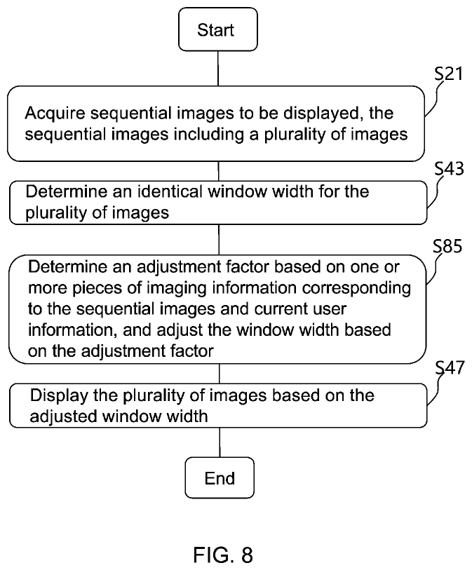

FIG. 8 shows a flowchart of an image display method of another embodiment of the present invention. As shown in FIG. 8, the image display method of this embodiment includes step S21, step S43, step S85, and step S47. In step S85, an adjustment factor is determined based on one or more pieces of imaging information corresponding to the sequential images and current user information, and the window width is adjusted based on the adjustment factor. In this case, the determined adjustment factor may at least include a component related to the imaging information and a component related to the user information. For example, in this step, mathematical computation may be further performed on the adjustment factor determined based on the imaging information and the adjustment factor determined based on the user information to obtain an overall adjustment factor, and the window width may be adjusted by using the overall adjustment factor. Alternatively, the window width may be first adjusted based on either the imaging information or the user information to obtain an intermediate value of the window width, and then the intermediate value may be further adjusted based on the other of these two to obtain an ultimate window width.

Further, a third deep learning network may be utilized to obtain the adjustment factor in step S85. For example, the current user information and the imaging information are input into a predetermined third deep learning network, an adjustment factor is output by the third deep learning network, and the window width determined in step S43 is adjusted based on the adjustment factor.

Data training may be performed by using the following exemplary method to obtain the third deep learning network described above.

In one step, ideal window width adjustment factors for a plurality of image sequences are acquired. For example, initial window widths may be first determined for the respective image sequences, then adjustment factors used by a plurality of users for adjusting the initial window widths are acquired, so that each of the image sequences has a plurality of ideal display brightness values for the plurality of users, and then a plurality of window width adjustment factors corresponding to the respective image sequences are calculated based on adjusted window widths and the initial window widths, wherein the plurality of window width adjustment factors correspond to multiple pieces of user information, respectively.

In another step, a plurality of window width adjustment factors of a plurality of image sequences are used as an output dataset, the imaging information and user information corresponding to the plurality of image sequences are used as an input dataset, and a suitable machine learning network is selected to perform machine learning, so as to assign a network parameters associated with the input dataset and the output dataset to the machine learning network.

The trained third deep learning network enables, when displaying an image sequence, acquisition of a window width adjustment factor capable of matching the imaging information so as to meet a display requirement of clinical diagnosis while also meeting a personal user preference.

In an embodiment of the present invention, the window level is determined as a median value of the window width.

As discussed herein, the deep learning technology (also referred to as deep machine learning, hierarchical learning, deep structured learning, or the like) employs an artificial neural network for learning. The deep learning method is characterized by using one or a plurality of network architectures to extract or simulate data of interest. The deep learning method may be implemented using one or a plurality of processing layers (for example, an input layer, an output layer, a convolutional layer, a normalization layer, or a sampling layer, where processing layers of different numbers and functions may exist according to different deep learning network models), where the configuration and number of the layers allow a deep learning network to process complex information extraction and modeling tasks. Specific parameters (or referred to as “weight” or “bias”) of the network are usually estimated through a so-called learning process (or training process). The learned or trained parameters usually result in (or output) a network corresponding to layers of different levels, so that extraction or simulation of different aspects of initial data or the output of a previous layer usually may represent the hierarchical structure or concatenation of layers. Thus, processing may be performed layer by layer. That is, “simple” features may be extracted from input data for an earlier or higher-level layer, and then these simple features are combined into a layer exhibiting features of higher complexity. In practice, each layer (or more specifically, each “neuron” in each layer) may process input data as output data for representation using one or a plurality of linear and/or non-linear transformations (so-called activation functions). The number of the plurality of “neurons” may be constant among the plurality of layers or may vary from layer to layer.

As discussed herein, a training data set having known input values (for example, known imaging information of an image sequence, user information, etc.,) and known or expected output values (for example, known ideal window width adjustment factors) may be employed as part of initial training of a deep learning process that solves a specific problem. In this manner, a deep learning algorithm may process a known data set or training data set (in a supervised or guided manner or an unsupervised or unguided manner), until a mathematical relationship between initial data and an expected output is identified and/or a mathematical relationship between the input and output of each layer is identified and characterized. (Partial) input data is usually used, and a network output is created for the input data in the learning process. Afterwards, the created output is compared with the expected (target) output of the data set, and then a generated difference from the expected output is used to iteratively update network parameters (weight and offset). One such update/learning mechanism uses a stochastic gradient descent method to update a network parameter. Apparently, those skilled in the art should understand that other methods known in the art may also be utilized. Similarly, a separate validation data set may be used, where both an input and an expected target value are known, but only an initial value is provided to a trained deep learning algorithm, and then an output is compared with an output of the deep learning algorithm to validate prior training and/or prevent excessive training.

Based on the description above, an embodiment of the present invention may further provide a computer-readable storage medium in which computer-readable instructions are stored, and the computer-readable instructions are configured to control a magnetic resonance scanning system to perform the image display method according to any one of the embodiments described above. The computer-readable storage medium may be similar to the storage medium in the controller 120 in the system shown in FIG. 1.

As used herein, an element or step described as singular and preceded by the word “a” or “an” should be understood as not excluding such element or step being plural, unless such exclusion is explicitly stated. Furthermore, references to “one embodiment” of the present invention are not intended to be interpreted as excluding the existence of additional embodiments that also incorporate the recited features. Moreover, unless explicitly stated to the contrary, embodiments “comprising,” “including,” or “having” an element or a plurality of elements having a specific property may include additional elements that do not have such property. The terms “including” and “in which” are used as the plain-language equivalents of the respective terms “comprising” and “wherein”. Furthermore, in the appended claims, the terms “first”, “second,” “third” and so on are used merely as labels, and are not intended to impose numerical requirements or a specific positional order on their objects.

This written description uses examples to disclose the present invention, including the best mode, and also to enable those of ordinary skill in the relevant art to implement the present invention, including making and using any devices or systems and performing any incorporated methods. The patentable scope of the present invention is defined by the claims, and may include other examples that occur to those skilled in the art. Such other examples are intended to be within the scope of the claims if they have structural elements that do not differ from the literal language of the claims, or if they include equivalent structural elements without substantial differences from the literal language of the claims.

1. An image display method for a magnetic resonance system, the method comprising:
acquiring sequential images to be displayed, the sequential images comprising a plurality of images;
determining an identical window width for the plurality of images; and
displaying the plurality of images of the sequential images based on the window width.

2. The method according to claim 1, wherein the determining an identical window width for the plurality of images comprises:
acquiring a sorting result of pixel values of the plurality of images; and
determining the window width based on a pixel value at a preset ordinal position in the sorting result or a plurality of pixel values within a preset ordinal position range.

3. The method according to claim 2, wherein the determining an identical window width for the plurality of images comprises:
determining the window width based on a pixel value at an intermediate ordinal position in the sorting result or a plurality of pixel values within an intermediate ordinal position range containing the intermediate ordinal position.

4. The method according to claim 1, wherein the determining the window width based on the plurality of pixel values within the intermediate ordinal position range comprises: determining an average of the plurality of pixel values as the window width.

5. The method according to claim 1, further comprising: determining an adjustment factor, and adjusting the window width based on the adjustment factor, wherein the displaying the plurality of images of the sequential images based on the identical window width comprises: displaying the plurality of images based on the adjusted window width.

6. The method according to claim 5, wherein the determining an adjustment factor comprises: determining the adjustment factor based on one or more pieces of imaging information corresponding to the sequential images.

7. The method according to claim 6, wherein the one or more pieces of imaging information comprise one or more of an imaging site, a scan sequence, a scan plane, an echo time, an inversion time, and a repetition time configured by the magnetic resonance system when generating the sequential images.

8. The method according to claim 7, wherein the determining an adjustment factor comprises: inputting the one or more pieces of imaging information into a predetermined deep learning network, and outputting the adjustment factor through the deep learning network.

9. The method according to claim 5, wherein the step of determining the adjustment factor comprises:
determining user information for displaying the sequential images; and
determining a corresponding adjustment factor based on the determined user information.

10. The method according to claim 5, wherein the step of determining the adjustment factor comprises:
determining user information for displaying the sequential images; and
inputting the determined user information into a predetermined second deep learning network, and outputting the adjustment factor through the second deep learning network.

11. A magnetic resonance system, comprising:
a scanner configured to generate sequential images by performing magnetic resonance scanning on an imaging site, the sequential images comprising a plurality of images;
a processor configured to acquire the sequential images and determine an identical window width for the plurality of images; and
a display unit displaying the plurality of images of the sequential images based on the identical window width.

12. A computer-readable storage medium for storing computer-readable instructions, wherein the computer-readable instructions are configured to perform the image display method according to claim 1.
